(19) United States
(12) Patent Application Publication
(10) Pub. No.: US 2024/0174601 A1
(43) Pub. Date: May 30, 2024

(54) COMPOSITION COMPRISING HETEROCYCLIC COMPOUND AND 4,4'-METHYLENE DIPHENYL DIISOCYANATE

(71) Applicant: BASF SE, Ludwigshafen am Rhein (DE)

(72) Inventors: Yang YANG, Shanghai (CN); Xiao Yu SUN, Shanghai (CN); Dong LIANG, Shanghai (CN); Stefan BOKERN, Lemfoerde (DE); Johannes BARTH, Shanghai (CN); Peter DEGLMANN, Ludwigshafen am Rhein (DE)

(21) Appl. No.: 18/282,281

(22) PCT Filed: Mar. 8, 2022
(86) PCT No.: PCT/EP2022/055936

§ 371 Date: Sep. 15, 2023

(30) Foreign Application Priority Data
Mar. 19, 2021 (WO) PCT/CN2021/081788

(51) Int. Cl. C07C 263/18; C07C 265/12; C07D 207/12; C07D 233/02; C07D 275/03

(52) U.S. Cl. CPC C07C 263/18; C07C 265/12; C07D 207/12; C07D 233/02; C07D 275/03

(57) ABSTRACT

This disclosure relates to a composition comprising (a) 4,4′-methylene diphenyl diisocyanate (4,4′-MDI), and (b) at least one heterocyclic compound comprising as ring moiety at least one structure of formula (I) as defined in the description. The heterocyclic compound can suppress the formation of 4,4′-MDI dimer and thus prevent the formation of insoluble solids and extend the shelf life of 4,4′-MDI.

### TECHNOLOGY FIELD

The present invention relates to a stable composition comprising 4,4′-methylene diphenyl diisocyanate (4,4′-MDI) and at least one heterocyclic compound and use of the heterocyclic compound for stabilizing 4,4′-methylene diphenyl diisocyanate.

### BACKGROUND

4,4′-Methylene Diphenyl Diisocyanate (4,4′-MDI) is one of the most industrially relevant isocyanates. However, it suffers from its tendency to dimerize and this has long been a challenge for the industry. Dimerization is an undesirable occurrence in the industry and has negative impacts on the product quality and application. Excess dimer formation will result in turbidity or precipitation. This leads to limitations of the transportation distance and storage time.

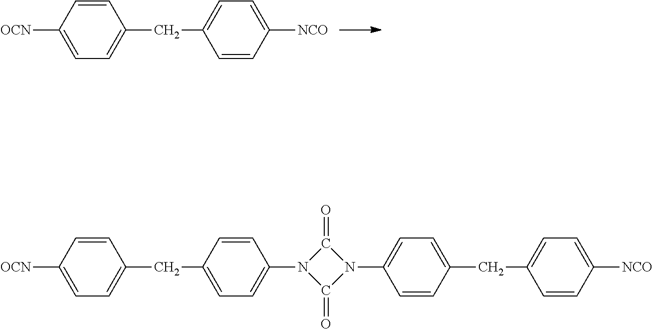

To minimize the dimerization effect and improve the product quality, there are two temperature windows to store and ship 4,4′-MDI:
(1) It can be stored and shipped as a liquid. This requires a precise temperature control within the range of 40-45° C. If the product is kept outside this range, the product quality will degrade quickly because of dimerization. Above 45° C., dimerization increases rapidly with the elevated temperature. Below 40° C., the product freezes and the dimerization is considerably promoted in the solid state. The advantage of a liquid state is the convenience to handle and use it directly after receiving it. However, even if it is handled at the proper condition, the dimer concentration still keeps increasing and at some point exceeds its saturation point (usually after several weeks).
(2) It can also be shipped as a solid. The solid must be kept at a very low temperature (generally lower than 0° C.). However, the customer needs to melt the solid before use. The melting process must be rapid and homogeneous. Long-time heating or partial overheating will generate a lot of dimers. Even if the melting process is handled properly, it is still recommended to use the product as soon as possible because a high concentration of dimer may be generated during the heating process.

Therefore, there is a strong need to use inhibitors to slower down the dimerization and extend the shelf life of 4,4′-MDI.

### SUMMARY OF THE INVENTION

It is an object of the invention to provide a composition comprising 4,4′-MDI and a heterocyclic compound, wherein the heterocyclic compound can suppress formation of 4,4′-MDI dimer and thus prevent the formation of insoluble solids and extending the shelf life of 4,4′-MDI.

Another object of the present invention is to provide use of the heterocyclic compound for stabilizing 4,4′-MDI.

It has been surprisingly found that the above objects can be achieved by following embodiments:
1. A composition comprising
(a) 4,4′-methylene diphenyl diisocyanate (4,4′-MDI), and
(b) at least one heterocyclic compound comprising as ring moiety at least one structure of formula (I)

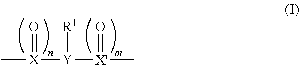

wherein X and X′ are each independently C or S;
n is 1 when X is C, or n is 1 or 2 when X is S;
m is 1 when X′ is C, or m is 1 or 2 when X′ is S;
Y is N or P; and
R¹ is selected from H, OH or halogen.
2. The composition according to item 1, wherein X and X′ are both C; or X and X′ are both S; or X is C and X′ is S; or X is S and X′ is C in the structure of formula (I) of the heterocyclic compound.
3. The composition according to items 1 or 2, wherein Y is N in the structure of formula (I) of the heterocyclic compound.
4. The composition according to any of items 1 to 3, wherein n is 2 when X is S in the structure of formula (I) of the heterocyclic compound.
5. The composition according to any of items 1 to 4, wherein m is 2 when X′ is S in the structure of formula (I) of the heterocyclic compound.
6. The composition according to any of items 1 to 5, wherein R¹ is H or halogen, for example halogen in the structure of formula (I) of the heterocyclic compound.
7. The composition according to any of items 1 to 6, wherein in addition to the heteroatoms in the structure of formula (I), the heterocyclic compound does not contain any heteroatoms as ring member, or contains 1 to 3, preferably 1 or 2 heteroatoms select from N, O or S as ring member.
8. The composition according to any of items 1 to 7, wherein the remaining ring of heterocyclic compound in addition to the structure of formula (I) is unsubstituted or is substituted by 1 to 5 substituents selected from OH, halogen, oxo group, C₁-C₁₀-alkyl, C₁-C₁₀-haloalkyl, C₃-C₁₀-cycloalkyl, C₃-C₁₀-halocycloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, phenyl or benzyl, wherein the alkyl moieties in the aforementioned C₁-C₁₀-alkyl, C₁-C₁₀-haloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl and halo-C₁-C₁₀-alkoxycarbonyl-C₁-C₁₀-alkyl may be interrupted by one or more nonadjacent groups which are selected from —O— and —S—.
9. The composition according to any of items 1 to 8, wherein the heterocyclic compound comprises 1 to 3, preferably 1 or 2 structures of formula (I).
10. The composition according to any of items 1 to 9, wherein the heterocyclic compound is a 5 to 14 membered heterocycle.
11. The composition according to any of items 1 to 10, wherein the heterocyclic compound is monocyclic or polycyclic, preferably monocyclic and contains 5 to 8 ring members, for example 5 ring members, or bicyclic and contains 8 to 14 ring members, for example 11 ring members.
12. The composition according to any of items 1 to 11, wherein the heterocyclic compound is selected from N-chlorosuccinimide, N-bromosuccinimide, Succinimide, 1,3-dichloro-5,5-dimethylimihydantoin, 1,3-dibromo-5,5-dimethylhydantoin, 5,5-dimethylhydantoin, N-Chlorosaccharin, N-Bromosaccharin, Saccharin or mixture thereof.
13. The composition according to any of items 1 to 12, wherein the composition comprises a heterocyclic compound (i) wherein R¹ is H and a heterocyclic compound (ii) wherein R¹ is halogen, preferably comprises 5,5-dimethylhydantoin as heterocyclic compound (i) and 1,3-Dichloro-5,5-dimethylhydantoin as heterocyclic compound (ii), or comprises succinimide as heterocyclic compound (i) and 1,3-Dichloro-5,5-dimethylhydantoin as heterocyclic compound (ii).
14. The composition according to any of items 1 to 13, wherein the heterocyclic compound is present in an effective amount for stabilizing 4,4′-MDI, preferably the amount of the heterocyclic compound is at least 10 ppm, more preferably in the range from 100 ppm to 5% by weight, in particular from 500 ppm to 3% by weight, based on the total weight of the composition.
15. The composition according to any of items 1 to 14, wherein the 4,4′-MDI is prepared via a phosgenation process or via a phosgene-free process.
16. Use of the heterocyclic compound as defined in any of items 1 to 14 for stabilizing 4,4′-MDI, preferably the heterocyclic compound is used to stabilize 4,4′-MDI in preservation or transportation.

The composition according to the present invention comprises a special heterocyclic compound, which can suppress the formation of 4,4′-MDI dimer and thus prevent the formation of insoluble solids and extend the shelf life of 4,4′-MDI.

### EMBODIMENT OF THE INVENTION

The undefined article “a”, “an”, “the” means one or more of the species designated by the term following said article.

In the context of the present disclosure, any specific values mentioned for a feature (comprising the specific values mentioned in a range as the end point) can be recombined to form a new range.

One aspect of the present invention is directed to a composition comprising
(a) 4,4′-methylene diphenyl diisocyanate (4,4′-MDI), and
(b) at least one heterocyclic compound comprising as ring moiety at least one structure of formula (I)

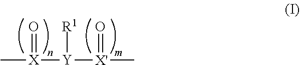

wherein X and X′ are each independently C or S;
n is 1 when X is C, or n is 1 or 2 when X is S;
m is 1 when X′ is C, or m is 1 or 2 when X′ is S;
Y is N or P; and
R¹ is selected from H, OH or halogen.

As component (b), the composition according to the present invention comprises at least one heterocyclic compound comprising as ring moiety at least one structure of formula (I).

The heterocyclic compound as component (b) can suppress the formation of 4,4′-MDI dimer, i.e., prevent the dimerization of 4,4′-MDI and thus is also called as inhibitor in this disclosure.

According to the present invention, X and X′ in the structure of formula (I) can be the same or different, for example X and X′ can be both C; or X and X′ can be both S; or X is C and X′ is S; or X is S and X′ is C in the structure of formula (I) of the heterocyclic compound.

In the structure of formula (I) of the heterocyclic compound, Y can be N or P, preferably N.

The value of n and m relates to X and X′, respectively. If X and X′ are C, then n and m is 1. If X and X′ are S, then n and m can be 1 or 2. In an embodiment, n is 2 when X is S. In an embodiment, m is 2 when X′ is S.

According to the present invention, R¹ can be selected from H, OH or halogen, for example H or halogen, for example halogen, such as chlorine, bromine and iodine. In a preferred embodiment, R¹ is H, chlorine or bromine, in particular bromine.

In a preferred embodiment, the variables in the structure of formula (I) have the following meaning:
X and X′ are each independently C or S;
n is 1 when X is C, or n is 1 or 2 when X is S;
m is 1 when X′ is C, or m is 1 or 2 when X′ is S;
Y is N; and
R¹ is selected from H, OH or halogen, preferably H or halogen, more preferably halogen;
or
X and X′ are each independently C or S;
n is 1 when X is C, or n is 1 or 2 when X is S;
m is 1 when X′ is C, or m is 1 or 2 when X′ is S;
Y is P; and
R¹ is selected from H, OH or halogen, preferably H or halogen, more preferably halogen;
or
X and X′ are each independently C or S;
n is 1;
m is 1;
Y is N; and
R¹ is selected from H, OH or halogen, preferably H or halogen, more preferably halogen;
or
X and X′ are each independently C or S;
n is 1 when X is C, or n is 2 when X is S;
m is 1 when X′ is C, or m is 2 when X′ is S;
Y is N; and
R¹ is selected from H, OH or halogen, preferably H or halogen, more preferably halogen;
or
X and X′ are C;
n is 1;
m is 1;
Y is N or P; and
R¹ is selected from H, OH or halogen, preferably H or halogen, more preferably halogen;
or
X and X′ are S;
n is 1 or 2;
m is 1 or 2;
Y is N or P; and
R¹ is selected from H, OH or halogen, preferably H or halogen, more preferably halogen;
or
X and X′ are C;
n is 1;
m is 1;
Y is N; and
R¹ is selected from H, OH or halogen, preferably H or halogen, more preferably halogen;
or
X and X′ are C;
n is 1;
m is 1;
Y is P; and
R¹ is selected from H, OH or halogen, preferably H or halogen, more preferably halogen;
or
X and X′ are S;
n is 1 or 2;
m is 1 or 2;
Y is N; and
R¹ is selected from H, OH or halogen, preferably H or halogen, more preferably halogen;
or
X and X′ are S;
n is 1 or 2;
m is 1 or 2;
Y is P; and
R¹ is selected from H, OH or halogen, preferably H or halogen, more preferably halogen.

According to the present invention, in addition to the heteroatoms in the structure of formula (I), the heterocyclic compound does not contain any heteroatoms as ring member, or contains 1 to 3, preferably 1 or 2 heteroatoms (additional heteroatom(s)) select from N, O or S as ring member. For example, the heterocyclic compound can contain 1 or 2 additional heteroatoms selected from N, O or S, especially N.

In the heterocyclic compound, the additional heteroatom(s) can be directly connected with the structure of formula (I) or be connected with the structure of formula (I) via one or more carbon atoms. In a preferred embodiment, the additional heteroatom (s), preferably N is directly connected with the structure of formula (I).

The remaining ring of heterocyclic compound in addition to the structure of formula (I) can be unsubstituted or substituted. For example, the remaining ring can be substituted by 1 to 5, preferably 1 to 3 substituents selected from OH, halogen, oxo group, C₁-C₁₀-alkyl, C₁-C₁₀-haloalkyl, C₃-C₁₀-cycloalkyl, C₃-C₁₀-halocycloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, phenyl or benzyl; preferably halogen, oxo group, C₁-C₁₀-alkyl or C₁-C₁₀-haloalkyl; wherein the alkyl moieties in the aforementioned C₁-C₁₀-alkyl, C₁-C₁₀-haloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl and halo-C₁-C₁₀-alkoxycarbonyl-C₁-C₁₀-alkyl may be interrupted by one or more (for example 1 to 8 or 1 to 5 or 1 to 4) nonadjacent groups which are selected from —O— and —S—.

In the context of the present disclosure, the organic moieties mentioned in the definitions of the variables are—like the term halogen—collective terms for individual listings of the individual group members. The prefix Cₙ-Cₘ indicates in each case the possible number of carbon atoms in the group.

“Halogen” or “halo” will be taken to mean fluoro, chloro, bromo and iodo.

The term “C₁-C₁₀-alkyl” (preferably C₁-C₆-alkyl) as used herein refers to a branched or unbranched saturated hydrocarbon group having 1 to 10 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “C₁-C₁₀-haloalkyl” (preferably C₁-C₆-haloalkyl) as used herein refers to a straight-chain or branched alkyl group having 1 to 10 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “C₁-C₁₀-alkoxy” (preferably C₁-C₆-alkoxy) and “C₁-C₁₀-alkylthio” (preferably C₁-C₆-alkylthio) refer to straight-chain or branched alkyl groups having 1 to 10 carbon atoms (as mentioned above) bonded through oxygen or sulfur linkages, respectively, in any position in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “C₁-C₁₀-haloalkoxy” (preferably C₁-C₆-haloalkoxy) and “C₁-C₁₀-haloalkylthio” (preferably C₁-C₆-haloalkylthio) refer to straight-chain or branched alkyl groups having 1 to 10 carbon atoms (as mentioned above) bonded through oxygen or sulfur linkages, respectively, in any position in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like.

The term “C₃-C₁₀-cycloalkyl” as used herein refers to a monocyclic 3- to 10-membered saturated carbon atom ring, e.g., cyclopropyl, cyclobutyl, cyclopentyl, cyclohexyl, cycloheptyl, cyclooctyl and cyclodecyl. Example includes C₅-C₇-cycloalkyl.

The term “C₃-C₁₀-halocycloalkyl” as used herein refers to a monocyclic 3- to 10-membered saturated carbon atom ring, e.g. cyclopropyl, cyclobutyl, cyclopentyl, cyclohexyl, cycloheptyl, cyclooctyl and cyclodecyl, where some or all of the hydrogen atoms in these groups may be replaced by halogen atoms as mentioned above, for example chloro-, dichloro- and trichlorocyclopropyl, fluoro-, difluoro- and trifluorocyclopropyl, chloro-, dichloro-, trichloro, tetrachloro-, pentachloro- and hexachlorocyclohexyl and the like. Example includes C₅-C₇-halocycloalkyl.

The term “C₁-C₁₀-alkylcarbonyl” as used herein refers to C₁-C₁₀-alkyl which is bound to the remainder of the molecule via a carbonyl group. Examples include CO—CH₃, CO—C₂H₅, CO—CH₂-C₂H₅, CO—CH(CH₃)₂, n-butylcarbonyl, CO—CH(CH₃)—C₂H₅, CO—CH₂—CH(CH₃)₂, CO—C(CH₃)₃, n-pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, n-hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl. Example includes C₁-C₆-alkylcarbonyl.

The term “C₁-C₁₀-alkoxycarbonyl” as used herein refers to C₁-C₁₀-alkoxy which is bound to the remainder of the molecule via a carbonyl group. Examples include CO—OCH₃, CO—OC₂H₅, CO—OCH₂-C₂H₅, CO—OCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl. Example includes C₁-C₆-alkoxycarbonyl.

In a preferred embodiment, the heterocyclic compound contains a nitrogen atom as ring member in addition to the heteroatoms in the structure of formula (I) and the nitrogen atom is substituted by a substituent selected from halogen, C₁-C₁₀-alkyl or C₁-C₁₀-haloalkyl, more preferably halogen, in particular bromine and chlorine.

The heterocyclic compound can comprise 1 to 3, preferably 1 or 2 structures of formula (I).

The heterocyclic compound according to the present invention can be a 5 to 14 membered heterocycle. The heterocyclic compound can be monocyclic or polycyclic, preferably monocyclic and contains 5 to 8, preferably 5 or 6 ring members, more preferably 5 ring members, or bicyclic and contains 8 to 14, preferably 9 to 12 ring members, more preferably 11 ring members. The heterocyclic compound can be a fused bicyclic system, which contains a 5- or 6-membered heterocyclic ring comprising the structure of formula (I) and a fused saturated or unsaturated, 5- or 6-membered carbocycle or a fused saturated or unsaturated, 5- or 6-membered heterocycle having 1, 2 or 3 heteroatoms, selected from 0, S or N as ring member. In a preferred embodiment, the heterocyclic compound is a fused bicyclic system, which contains a 5- or 6-membered heterocyclic ring comprising the structure of formula (I) and a fused phenyl ring or a fused 5- or 6-membered aromatic heterocycle having 1, 2 or 3 heteroatoms, selected from 0, S or N as ring member. In a preferred embodiment, heterocyclic compound is monocyclic and contains 5 ring members, or the heterocyclic compound is a fused bicyclic system, which contains a 5-membered heterocyclic ring comprising the structure of formula (I) and a fused phenyl ring.

The specific examples of the heterocyclic compound can include:

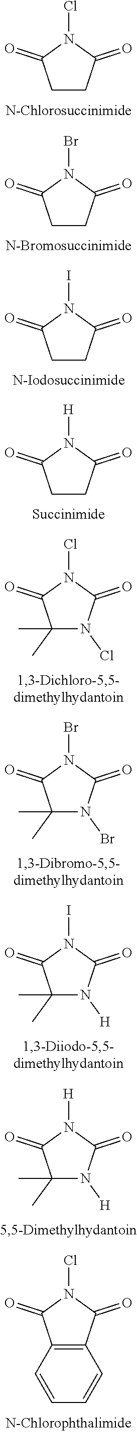

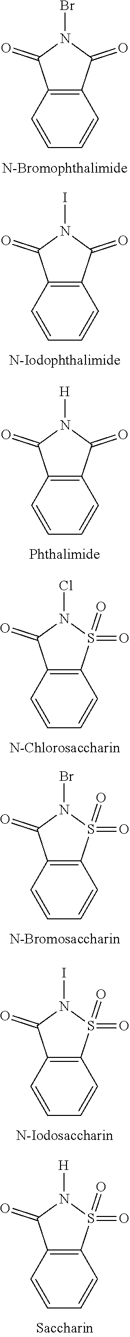

According to the present invention, the heterocyclic compound can be present in an effective amount for stabilizing 4,4′-MDI, preferably the amount of the heterocyclic compound is at least ppm, for example at least 50 ppm, at least 100 ppm, at least 150 ppm, at least 200 ppm, at least 300 ppm, at least 500 ppm, at least 800 ppm, at least 1000 ppm, at least 2000 ppm, at least 3000 ppm, at least 4000 ppm, or at least 5000 ppm, more preferably in the range from 100 ppm to 5% by weight, in particular from 500 ppm to 3% by weight or from 500 ppm to 2% by weight or from 500 ppm to 1% by weight, based on the total weight of the composition.

In an embodiment, the composition comprises a combination containing at least two, for example two or three or more heterocyclic compounds of the present invention.

In a preferred embodiment, the composition comprises a heterocyclic compound (i) wherein R¹ is H and a heterocyclic compound (ii) wherein R¹ is halogen, preferably comprises 5,5-dimethylhydantoin as heterocyclic compound (i) and 1,3-Dichloro-5,5-dimethylhydantoin as heterocyclic compound (ii), or comprises succinimide as heterocyclic compound (i) and 1,3-Dichloro-5,5-dimethylhydantoin as heterocyclic compound (ii).

The ratio of heterocyclic compound (i) to heterocyclic compound (ii) can be in the range from 100:1 to 1:100, preferably from 50:1 to 1:50, more preferably 1:10 to 10:1, for example 8:1, 5:1, 3:1, 2:1, 1:1, 1:2, 1:3, 1:5 or 1:8.

According to the present invention, the composition comprises 4,4′-MDI as component (a). 4,4′-MDI can be prepared via a phosgenation process or via a phosgene-free process.

The amount of 4,4′-MDI can be at least 80 wt. %, at least 90 wt. %, at least 95 wt. %, at least 96 wt. %, at least 97 wt. %, at least 98 wt. % or at least 99 wt. %, or even at least 99.5 wt. %, based on the total weight of the composition.

In a preferred embodiment, the composition can have a temperature in the range from 40 to 180° C., for example 41° C., 42° C., 43° C., 44° C., 45° C., 46° C., 47° C., 48° C., 49° C., 50° C., 60° C., 80° C., 100° C., 120° C., 150° C., 180° C., preferably from 41 to 120° C. or from 41 to 80° C., more preferably from 41 to 46° C.

After 28 days at 42° C., the content of the dimer in the composition of the present invention is lower than the content of the dimer in an otherwise identical comparative composition only without said heterocyclic compound. For example, after 28 days at 42° C., the content of the dimer in the composition of the present invention can be lower than the content of dimer in the comparative composition only without said heterocyclic compound by at least 5%, preferably at least 8% or 10%, more preferably at least 15% or 20%), especially at least 25%, based on the dimer content in the comparative composition. For example, if the dimer contents in the composition of the present invention and comparative composition are “a” and “b”, respectively, after 28 days at 42° C., then the content of the dimer in the composition of the present invention is lower than the content in the comparative composition without said heterocyclic compound by (b−a)×100%/b.

The content of dimer can be determined by the quantitative infrared spectroscopy analysis according to Standard ASTM D8036-16 issued by ASTM International, West Conshohocken, Pennsylvania, United States.

The composition of the present invention can be prepared by adding the heterocyclic compound as defined in the present disclosure to 4,4′-MDI liquid. Preferably, 4,4′-MDI has a temperature mentioned for the composition.

A further aspect of this disclosure relates to use of the heterocyclic compound as defined in the present disclosure for stabilizing 4,4′-MDI.

According to the present invention, the heterocyclic compound can be used in an amount as mentioned for the composition of the present invention.

According to the present invention, the heterocyclic compound can be used to stabilize 4,4′-MDI in preservation or transportation.

### EXAMPLES

The present invention is further illustrated by the following examples, which are set forth to illustrate the present invention and is not to be construed as limiting thereof. Unless otherwise noted, all parts and percentages are by weight.

Materials

4,4′-MDI: 4,4′-methylene diphenyl diisocyanate, supplied by BASF,

Methods

Content of 4.4′-MDI dimer: the content of dimer was determined by the quantitative infrared spectroscopy analysis according to Standard ASTM D8036-16 issued by ASTM International, West Conshohocken, Pennsylvania, United States.

### Examples 1 to 9 and Comparative Examples 1 and 2

In examples 1 to 9, one heterocyclic compound (inhibitor) is used as component (b). Each component (b) in an amount as shown in table 1 was added to 4,4′-MDI to obtain the composition according to the present invention. Comparative example 1 was a blank and no component (b) was added. In comparative example 2, benzoyl chloride was used. The compositions of examples 1 to 9 and comparative example 2 and 4,4′-MDI of comparative example 1 were stored at 42° C. The initial contents of 4,4′-MDI dimer and dimer contents after 14 days and 28 days were also shown in table 1.

TABLE 1
Experiments using one inhibitor

| | | | initial dimer | dimer content | dimer content |
|---|---|---|---|---|---|
| | | Percentage | content | after 14 | after 28 |
| | inhibitors | of the inhibitors | (wt. %) | days (wt. %) | days (wt. %) |
| Comparative | no | | 0.26 | 0.34 | 0.43 |
| example 1 | | | | | |
| Comparative | benzoyl chloride | 0.5% | 0.26 | 0.35 | 0.43 |
| example 2 | | | | | |
| Example 1 | N- | 0.1% | 0.26 | 0.32 | 0.37 |
| | Chlorosuccinimide | | | | |
| Example 2 | N-Chlorosuccinimide | 0.5% | 0.26 | 0.31 | 0.35 |
| Example 3 | N-Bromosuccinimide | 0.5% | 0.26 | 0.26 | 0.27 |
| Example 4 | Succinimide | 0.5% | 0.26 | 0.30 | 0.34 |
| Example 5 | 1,3-Dichloro-5,5- | 0.1% | 0.26 | 0.32 | 0.38 |
| | dimethylhydantoin | | | | |
| Example 6 | 1,3-Dichloro-5,5- | 0.5% | 0.26 | 0.30 | 0.34 |
| | dimethylhydantoin | | | | |
| Example 7 | 1,3-Dibromo-5,5- | 0.5% | 0.26 | 0.26 | 0.27 |
| | dimethylhydantoin | | | | |
| Example 8 | 5,5- | 0.5% | 0.26 | 0.28 | 0.30 |
| | dimethylhydantoin | | | | |
| Example 9 | Saccharin | 0.5% | 0.26 | 0.33 | 0.39 |

As can be seen, the addition of heterocyclic compound as component (b) in examples 1 to 9 suppressed the formation of 4,4′-MDI dimer comparing with the comparative example 1 without heterocyclic compound of the present invention. There was no finding of stabilizing effect in comparative example 2 by adding Benzoyl chloride.

### Examples 10 and 11

In examples 10 and 11, a combination of two heterocyclic compounds (inhibitor) were used as component (b). Each component (b) in an amount as shown in table 2 was added to 4,4′-MDI to obtain the compositions according to the present invention. Comparative example 1 was a blank and no component (b) was added. The compositions of examples 10 and 11 and 4,4′-MDI of comparative example 1 were stored at 42° C. The initial contents of 4,4′-MDI dimer and dimer contents after 14 days and 28 days were also shown in table 2.

TABLE 2
Experiments using two inhibitors.

| | | initial dimer | dimer content | dimer content |
|---|---|---|---|---|
| | | content | after 14 days | after 28 days |
| | inhibitors | (wt. %) | (wt. %) | (wt. %) |
| Comparative | no | 0.26 | 0.34 | 0.43 |
| example 1 | | | | |
| Example 10 | 0.06% 1,3-Dichloro-5,5- | 0.26 | 0.30 | 0.34 |
| | dimethylhydantoin | | | |
| | and 0.09% 5,5- | | | |
| | dimethylhydantoin | | | |
| Example 11 | 0.06% 1,3-Dichloro-5,5- | 0.26 | 0.32 | 0.38 |
| | dimethylhydantoin and | | | |
| | 0.09% Succinimide | | | |

As can be seen, the formation of 4,4′-MDI dimer is also suppressed by using two heterocyclic compounds.

1.-16. (canceled)

17. A composition comprising
(a) 4,4′-methylene diphenyl diisocyanate (4,4′-MDI), and
(b) at least one heterocyclic compound comprising as ring moiety at least one structure of formula (I) 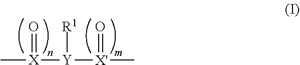
wherein X and X′ are each independently C or S;
n is 1 when X is C, or n is for 2 when X is S;
m is 1 when X′ is C, or m is 1 or 2 when X′ is S;
Y is N or P; and
R¹ is selected from H, OH or halogen.

18. The composition according to claim 17, wherein X and X′ are both C; or X and X′ are both S; or X is C and X′ is S; or X is S and X′ is C in the structure of formula (I) of the heterocyclic compound.

19. The composition according to claim 17, wherein Y is N in the structure of formula (I) of the heterocyclic compound.

20. The composition according to claim 17, wherein n is 2 when X is S in the structure of formula (I) of the heterocyclic compound.

21. The composition according to claim 17, wherein m is 2 when X′ is S in the structure of formula (I) of the heterocyclic compound.

22. The composition according to claim 17, wherein R¹ is H or halogen, for example halogen in the structure of formula (I) of the heterocyclic compound.

23. The composition according to claim 17, wherein in addition to the heteroatoms in the structure of formula (I), the heterocyclic compound does not contain any heteroatoms as ring member.

24. The composition according to claim 17, wherein the remaining ring of heterocyclic compound in addition to the structure of formula (I) is unsubstituted or is substituted by 1 to 5 substituents selected from OH, halogen, oxo group, C₁-C₁₀-alkyl, C₁-C₁₀-haloalkyl, C₃-C₁₀cycloalkyl, C₃-C₁₀-halocycloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, phenyl or benzyl, wherein the alkyl moieties in the aforementioned C₁-C₁₀-alkyl, C₁-C₁₀-haloalkyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl and halo-C₁-C₁₀-alkoxycarbonyl-C₁-C₁₀-alkyl may be interrupted by one or more nonadjacent groups which are selected from —O— and —S—.

25. The composition according to claim 17, wherein the heterocyclic compound comprises 1 to 3 structures of formula (I).

26. The composition according to claim 17, wherein the heterocyclic compound is a 5 to 14 membered heterocycle.

27. The composition according to claim 17, wherein the heterocyclic compound is monocyclic or polycyclic, and contains 5 to 8 ring members, for example 5 ring members, or bicyclic and contains 8 to 14 ring members, for example 11 ring members.

28. The composition according to claim 17, wherein the heterocyclic compound is selected from N-chlorosuccinimide, N-bromosuccinimide, succinimide, 1,3-dichloro-5,5-dimethylimihydantoin, 1,3-dibromo-5,5-dimethylhydantoin, 5,5-dimethylhydantoin, N-Chlorosaccharin, N-Bromosaccharin, Saccharin or mixture thereof.

29. The composition according to claim 17, wherein the composition comprises a heterocyclic compound (i) wherein R¹ is H and a heterocyclic compound (ii) wherein R¹ is halogen, and 1,3-Dichloro-5,5-dimethylhydantoin as heterocyclic compound (ii), or comprises succinimide as heterocyclic compound (i) and 1,3-Dichloro-5,5-dimethylhydantoin as heterocyclic compound (ii).

30. The composition according to claim 17, wherein the heterocyclic compound is present in an effective amount for stabilizing 4,4′-MDI, based on the total weight of the composition.

31. The composition according to claim 17, wherein the 4,4′-MDI is prepared via a phosgenation process or via a phosgene-free process.

32. A method comprising providing the heterocyclic compound as defined in claim 17 and stabilizing 4,4′-MDI in preservation or transportation.
